(19) United States
(12) Patent Application Publication
(10) Pub. No.: US 2012/0245632 A1
(43) Pub. Date: Sep. 27, 2012

(54) BONE ANCHOR

(75) Inventors: Yung-Fang Tsai, Taichung City (TW); Wei-Kai Wang, Taichung City (TW); Yang-Hwei Tsuang, Taichung City (TW); Yi-Jie Kuo, Taichung City (TW)

(73) Assignee: INTAI TECHNOLOGY CORP., Taichung City (TW)

(21) Appl. No.: 13/225,423

(22) Filed: Sep. 3, 2011

(30) Foreign Application Priority Data
Mar. 24, 2011 (TW) 100205296

(51) Int. Cl. A61B 17/04

(52) U.S. Cl. 606/232

(57) ABSTRACT

A bone anchor comprises a screw body and a flange, which are joined together in a way to keep their rotation independent from each other. In implanting the screw body into a bone, the flange does not rotate to prevent twisting and/or snarling of the suture in wounded bone from occurring.

### BACKGROUND OF THE INVENTION

1. Field of the Invention

The present invention relates to surgical devices for assisting the recovery of bone injury, such as bone fracture or dislocation. Especially, the present invention relates to a bone anchor adapted to use with sutures for wounded bone.

2. Description of the Related Art

A technique of combining an anchor with sutures for wounded bone has been used in the surgical devices for assisting the recovery of bone injury, such as bone fracture or dislocation. Examples of said suture used for wounded bone reduction include seam thread, steel wire, retaining belt, and the like. In practice, the anchor is implanted into a healthy bone, while the sutures are connected to the anchor and then fastened to the wounded bone. The anchor and suture provide an auxiliary force in a restoring direction to reset the wounded bone, so as to maintain the wounded bone on correct anatomical position for faster and more effective recovery.

U.S. Pat. No. 6,319,270 disclosed a screw suture anchor, which has suture-receiving eyelet on the driving head of the screw. However, there are a few problems which should be mentioned for they may reduce the efficacy of the screw suture anchor. First, the number and caliber of sutures used will be limited because of the small pore size of the suture-receiving eyelet. Second, the driving head rotates simultaneously as the screw is turned into bone, which will result in the twisting and snarling of the suture. Third, the piercing angle at the edge margins between the threading hole and the driving head may damage or cut the suture.

### SUMMARY OF THE INVENTION

The object of this invention is to provide a bone anchor adapted to use with sutures for wounded bone. The bone anchor of the present invention is a screw bone anchor comprising a screw body and a flange, which are individual objects and joined together in a way to keep their independent rotating. The flange has threading holes to allow the sutures to pass through. Basing the jointing technique of the screw body to the flange, the flange can stay in location without rotating when the screw body is turned into the bone. This way, the twisting and/or snarling of the suture in the wounded bone can be avoided.

The jointing technique to maintain individual rotary freedom of the screw body and the flange also provides good bonding strength and stability to make the flange a stable basement for the wiring of the suture, and therefore enables the bone anchor and suture to provide an auxiliary force for effective resorting of wounded bone.

Additionally, the jointing technique described above can make the assembly of the screw body and the flange to exhibit an offset of rotating angle in a proper range, which allows the screw body and the flange to be adapted to various anatomical structures of bones and soft tissues for surgical implantation and fixation.

The present screw bone anchor can maximize the pore size of the suture-receiving eyelets while maintaining bonding strength of the screw body and the flange and keeping the structural rigidity of the flange to avoid limitations on the number and caliber of the suture in usage.

An arc radius (“R”) design is used for the edge margins of said suture-receiving eyelets to avoid any damage to the suture caused by right-angled rim.

The screw body of the present bone anchor possesses a self-tapping cutting end, which has a self-tapping cutting bit of a special shape. In an implanting operation, the screw body will be easily tapped into the bone tissue.

To achieve the purposes mentioned above, the bone anchor of this invention includes a screw body and a flange, which are joined together in a way to keep their individual rotary freedom. The flange has at least two suture-receiving eyelets formed through the thickness of flange, and the two suture-receiving eyelets are equiangularly positioned on the flange. The pore size of the two suture-receiving eyelets will be maximized while maintaining bonding strength of the screw body and the flange, and keeping the structural rigidity of the flange. Furthermore, the boundary edged between the suture-receiving eyelet and the flange is designed as an arc radius. The free end of the screw body is a self-tapping cutting end.

### BRIEF DESCRIPTION OF THE DRAWINGS

FIG. 1 is a three-dimensional exploded view showing the first bone anchor example of this invention.

FIG. 2 shows the assembly of the first bone anchor example of this invention.

FIG. 3 is a sectional view showing the assembly of the first bone anchor example of this invention.

FIG. 4 is a three-dimensional exploded view showing the second bone anchor example of this invention.

FIG. 5 shows the assembly of the second bone anchor example of this invention.

FIG. 6 is a sectional view showing the assembly of the second bone anchor example of this invention.

FIG. 7 is a three-dimensional exploded view showing the third bone anchor example of this invention.

FIG. 8 shows the assembly of the third bone anchor example of this invention.

FIG. 9 is a sectional view showing the assembly of the third bone anchor example of this invention.

FIG. 10 is a schematic diagram showing the offset of rotating angle in the bone anchor of the present invention.

### DETAILED DESCRIPTION OF THE INVENTION

The specific examples below are to be construed as merely illustrative, and not limitative of the remainder of the disclosure in any way whatsoever. Without further elaboration, it is believed that one skilled in the art can, based on the description herein, utilize the present invention to its fullest extent.

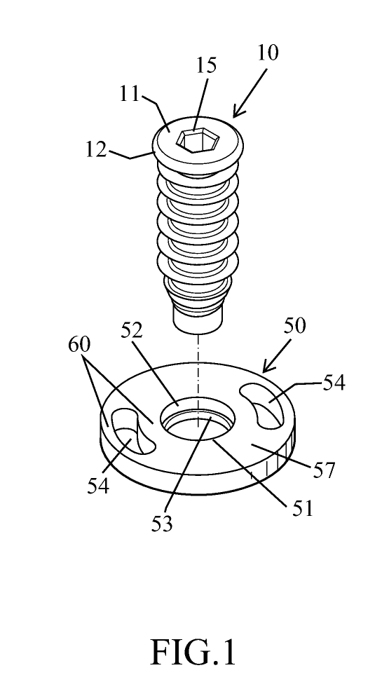

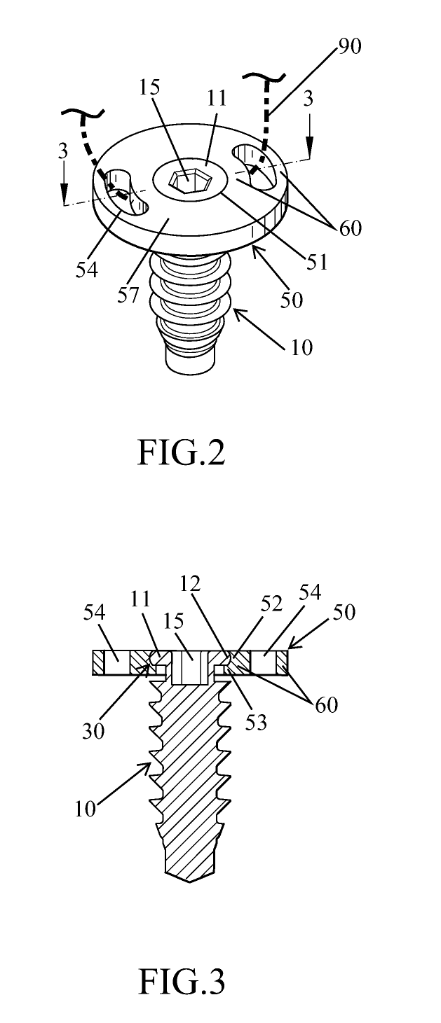

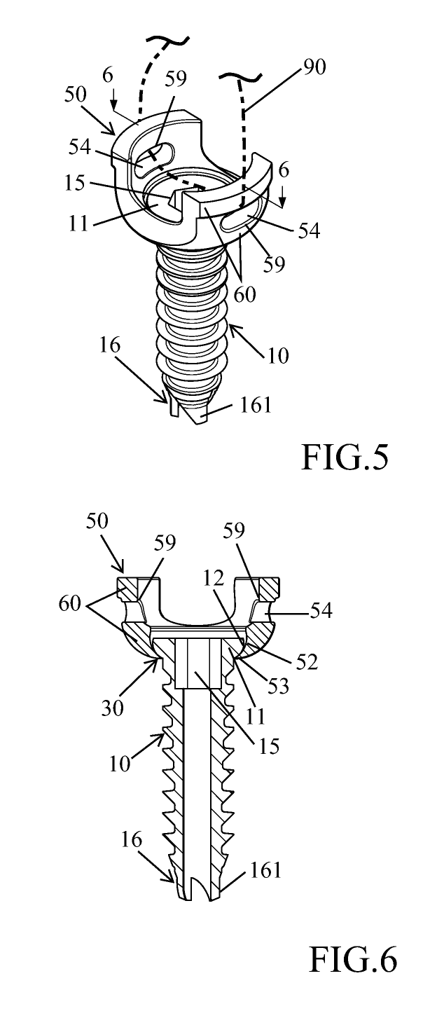

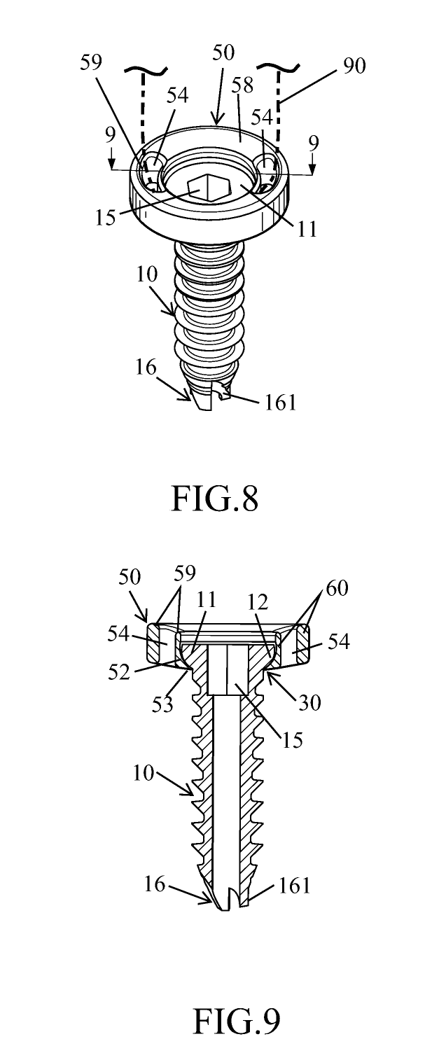

As shown in FIG. 1 to FIG. 9, the bone anchor of this invention comprises a screw body 10 and a flange 50, which are joined together coaxially in a combining structure 30. The combining structure 30 includes: a central hole 51 in the flange 50, an arcuate-concave side wall 52 surrounding the central hole 51, a supporting part 53 formed on the bottom end of the arcuate-concave side wall 52 as a protruding eave, and an arc convex shaped side wall 12 formed on the head 11 of the screw body 10. In combination, the screw body 10 is passed through the central hole 51 of the flange 50, the head 11 is supported by the supporting part 53, and the arc convex shaped side wall 12 is engaged with the arcuate-concave side wall 52.

The screw body 10 and the flange 50 are individual objects, joined together via the combining structure 30. The combining structure 30 provides individual rotary freedom to both screw body 10 and flange 50 after the combination. The head 11 of the screw body 10 has a hexagonal operation hole 15 for combining with an operative apparatus. When the rotating screw body 10 is drilled into bone driven by an operative apparatus, the flange 50 will not rotate by using the combining structure 30.

The flange 50 possesses at least two suture-receiving eyelets 54 formed therethrough. A suture 90 can be threaded and connected to the flange 50 through the suture-receiving eyelets 54. The two suture-receiving eyelets 54 are equiangularly spaced on the flange 50. The function of the combining structure 30 to keep the positioning of flange 50 independent from the rotation of the screw body 10 will keep the suture connected to the flange 50 at the defined threading location, and therefore prevent rotation, twisting and snarling from occurring. It is known that the suture 90 must be knotted onto a wounded bone, and provides an auxiliary force in restoring direction to reset the wounded bone to maintain the wounded bone on the correct anatomical position for faster and more effective recovery. When the suture 90 performs such functions described above, a tensile force is formed in the opposite direction to the flange 50. At this time, the combining structure 30 provides a sufficient anchoring force for the screw body 10 and the flange 50 to keep the flange 50 axially positioned on the screw body 10 and not disengaged from the screw body 10. This becomes a firm basis for the suture 90.

It is preferable to maximize the pore size of the suture-receiving eyelets 54 on the flange 50 for easier threading of suture 90 and connecting to the flange 50, and for increasing the number and caliber of sutures as needed for practical usage. The pore size will be maximized while maintaining bonding strength of the screw body and the flange and keeping the structural rigidity of the flange. In the embodiments of this invention, an appropriate cross section area 60 is provided between the suture-receiving eyelet 54 and the central hole 51 and outer side wall of the flange 50. This area is disposed to maintain the rigidity of this part, to compensate for the tractive force added on the flange 50 by the suture 90, and to avoid cracking caused by stress concentration. Besides, the suture-receiving eyelet 54 has an elongated shape to increase the area of orifice. In the embodiments of this invention, two suture-receiving eyelets are provided. It will be understood that the number of suture-receiving eyelets is not to be limited to two. In fact, three or four or even more suture-receiving eyelets may also be practicable.

An embodiment designed for protecting the suture 90 from damage by the suture-receiving eyelet 54 is shown in FIG. 6. In this embodiment, a design of arc radius is provided to the edge margins 59 of the suture-receiving eyelets 54 and the flange 50. This design of arc radius can avoid damages to the suture by acute angle.

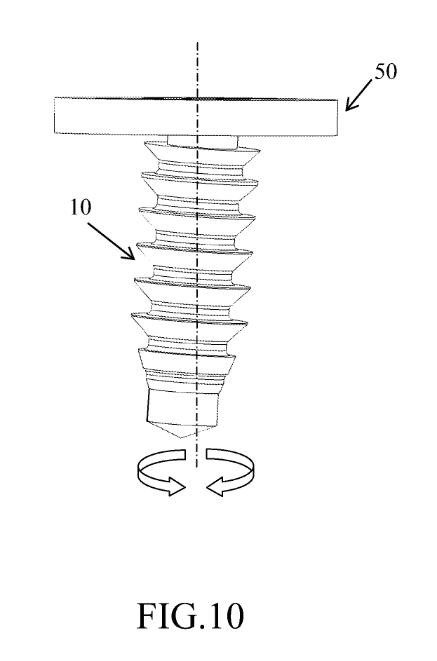

Another embodiment providing flexible angle adjustment to the flange 50 and the screw body 10 is shown in FIG. 10. The engagement of the ball convex shaped side wall 12 of the head 11 to the arcuate-concave side wall 52 of the central hole 51 in the flange 50 creates an offset of rotating angle in a proper range. The offset of rotating angle is determined by the radians of the arc convex shaped side wall 12 and the arcuate-concave side wall 52, which may be set in manufacture as the requirements for the anatomical structure of bones and soft tissues.

Besides, in the embodiments of this invention, a self-tapping cutting end 16 is provided to the free end of the screw body 10. The self-tapping cutting end may be established by various kinds of self-tapping cutting bit 161.

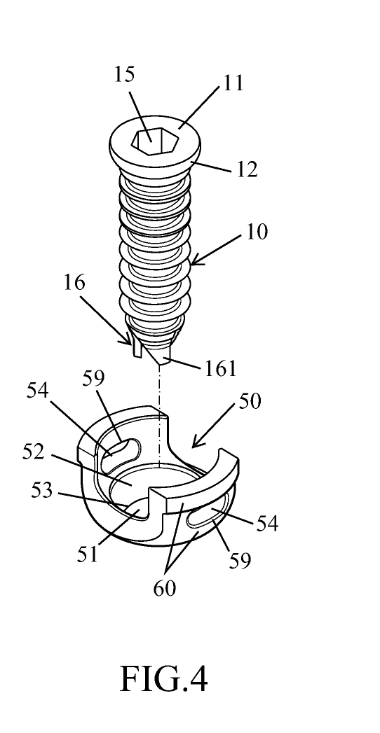

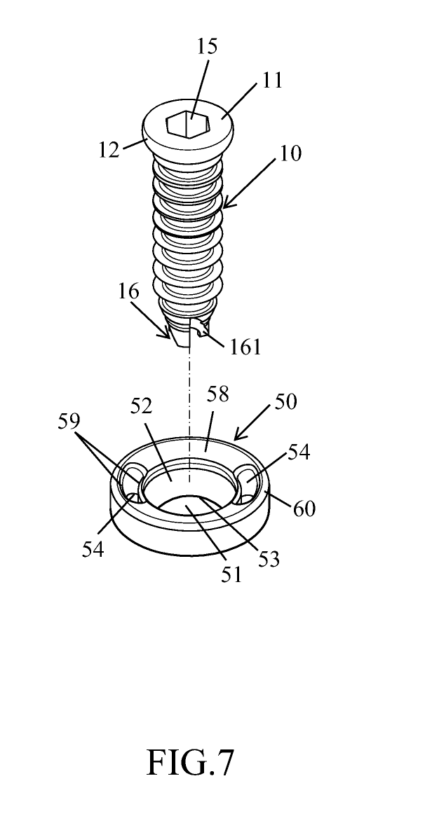

Three embodiments of present bone anchor are disclosed. These embodiments exhibit the technical characteristics described above with only difference on the functions of the flange 50. The flange 50 described in FIGS. 1 to 3 possesses a flat top surface 57, which is designed to have the head 11 of the screw body 10 terminated in a flat position corresponding to the top surface 57 of flange 50. The flange 50 described in FIGS. 4 to 6 has a U-shaped structure. And the flange 50 described in FIGS. 7 to 9 has an arcuate-concave top surface 58, wherein the position of the head 11 of the screw body 10 is lower than the lowest point of the top surface 58 of the flange 50 to provide a design of sunk head. The shape of flange 50 is determined according to the anatomical structure of bones and adjacent soft tissues in the operated region.

What is claimed is:

1. A bone anchor, including
a screw body;
a flange, having at least a suture-receiving eyelet; and
a combining structure to make the screw body and the flange joined together coaxially and to keep their individual rotary freedom.

2. The bone anchor of claim 1, wherein the combining structure includes a central hole set in the flange for combining with the head of the screw body, an arcuate-concave side wall in the central hole, and an arc convex shaped side wall of the head of the screw body, the ball convex shaped side wall is engaged to the ball cup shaped side wall.

3. The bone anchor of claim 2, further including a supporting part formed on the bottom end of the ball cup shaped side wall for supporting the head of the screw body.

4. The bone anchor of claim 2, wherein a structural cross section area is further provided between the suture-receiving eyelet and the central hole, and between the suture-receiving eyelet and the outer side wall of the flange to maintain the rigidity of the flange.

5. The bone anchor of claim 1, wherein the margin of the suture-receiving eyelet and the flange is designed as an arc radius.

6. The bone anchor of claim 1, wherein the flange possesses a flat top surface.

7. The bone anchor of claim 1, wherein the flange possesses an arcuate-concave top surface.

8. The bone anchor of claim 1, wherein the flange has a U-shaped structure.

9. The bone anchor of claim 1, wherein the screw body comprises a self-tapping cutting end.
